(19) United States
(12) Patent Application Publication
(10) Pub. No.: US 2010/0016744 A1
(43) Pub. Date: Jan. 21, 2010

(54) FETAL HEART RATE MONITORING SYSTEM

(75) Inventors: Brian C. Brost, Rochester, MN (US); Russell E. Bruhnke, Rochester, MN (US); Patrick E. Caskey, Rochester, MN (US); Stephen A. Contag, Rochester, MN (US); Dan Dragomir Daescu, Rochester, MN (US); Bruce A. Winter, Rochester, MN (US); Jonathon W. Lange, Plainview, MN (US)

Correspondence Address: FAEGRE & BENSON LLP;PATENT DOCKETING - INTELLECTUAL PROPERTY 23/24, 2200 WELLS FARGO CENTER, 90 SOUTH SEVENTH STREET, MINNEAPOLIS, MN, 55402-3901, US

(73) Assignee: Mayo Foundation for Medical Education and Research, Rochester, MN (US)

(21) Appl. No.: 12/297,016

(22) PCT Filed: Apr. 13, 2007
(86) PCT No.: PCT/US07/09247

§ 371 Date: Jun. 4, 2009

Related U.S. Application Data
(60) Provisional application No. 60/791,861, filed on Apr. 13, 2006.

(51) Int. Cl. A61B 5/0444

(52) U.S. Cl. 600/511

(57) ABSTRACT

A fetal heart rate monitoring system includes an ultrasonic transducer for transmitting a signal to a patient and for receiving a reflected heart beat monitoring signal. A monitoring system coupled to the ultrasonic transducer processes the heart beat monitoring signal and produces heart beat information representative of the monitored heart rate. A mechanical or electronic steering system coupled to the ultrasonic transducer controls the location of the signal transmitted to the patent and/or the location of the received heart beat monitoring signal. A control system coupled to the monitor and steering system controls the steering system as a function of the heart beat monitoring signal to optimize the quality of the heart beat information in the received heart beat monitoring signals.

### REFERENCE TO RELATED APPLICATION

This application claims the benefit of U.S. Provisional Application Ser. No. 60/791,861 filed Apr. 13, 2006 and entitled Fetal Heart Rate Monitoring System, which is incorporated herein by reference.

### FIELD OF THE INVENTION

The present invention relates to fetal heart rate monitoring systems. In particular, the invention is a system for controlling the position or orientation of a Doppler heart rate monitor as the fetus moves.

### BACKGROUND OF THE INVENTION

Few events are more distressing in the life of parents than the severe injury or death of a child before or during delivery. Fetal heart rate monitoring is commonly used to assess fetal well-being in the United States. Experience has shown that pregnancy is labeled as high-risk in a portion (about 20%) of these cases prior to labor, and a lesser portion (about 5-10%) during labor. Experience has also shown that these groups account for about 50% and 20-25%, respectively, of poor obstetric outcomes. Unfortunately, about 20% of perinatal morbidity and mortalities occur in women deemed to be a low risk during pregnancy.

It is evident that clinical outcomes can be improved by fetal monitoring. The assessment of fetal well-being can make use of a continuous signal from the fetal heart to assess beat-to-beat and long term changes. Fetal heart rate monitoring systems that can be used for this purpose are known and commercially available. One such system is the 50XM Intrapartum Fetal Monitor available from Philips. Systems of this type include a transducer for generating a generally unidirectional ultrasonic signal that is transmitted to and reflected back from the fetal heart. Beating-induced movements of the heart introduce what is known as the Doppler effect on the transmitted signal before it is reflected back to the transducer. The monitoring system processes the received signal to identify and generate information representative of the fetal heart rate.

The ultrasonic monitoring system described above generates and displays or presents several signals representative of the fetal heart beat. One of these signals is an audio signal representative of the beating heart. This signal can be presented to clinicians through a speaker.

The other is a quality level signal displayed by LEDs. The quality level signal and display is representative of the quality of the ultrasonic signal received by the transducer. The ability of the monitoring system to generate an accurate representation of the heart beat is dependant upon the accuracy by which the ultrasonic signal is directed or pointed to the heart, and the amount of the reflected signal that is received by the transducer (e.g., the signal to noise ratio in the reflected signal). For example, if the ultrasonic signal is not directed squarely at the heart, the reflected component that is received and processed will have reduced Doppler effect information resulting in a lower quality heart beat signal. The Philips 50XM monitoring system has three LEDs for indicating good, marginal and poor signal quality levels. The quality level signal can also be accessed from a terminal on the back of the monitoring system.

There remains a continuing need for improved fetal heart rate monitoring systems. In particular, there is a need for a system capable of continuously providing a high-quality signal even in the presence of maternal and fetal movements that occur during antenatal and labor assessment. A system of this type may decrease parental anxiety and enhance the efficiency of health care services delivery.

### SUMMARY OF THE INVENTION

The invention is a fetal heart rate monitoring system capable of continuously providing a high-quality signal, even in the presence of maternal and fetal movement. One embodiment of the invention includes a transducer for transmitting and/or receiving heart beat monitoring signals and a steering system for controlling the location of the transmitted and/or received heart beat monitoring signals. A monitoring system coupled to the transducer processes the received heart beat monitoring signals and produces heart beat information representative of a monitored heart beat. A control unit coupled to the transducer and the steering system processes the received heart beat monitoring signals and controls the steering system to optimize quality of the heart beat monitoring signals.

In another embodiment of the invention the control unit controls the steering system to optimize the quality of the heart beat information in the heart beat monitoring signals. The steering system can be either a mechanical system including a gimbal or an electronic system including an array of transducers or other structures or methods for steering the beam within the transducer.

### BRIEF DESCRIPTION OF THE DRAWINGS

FIG. 1 is a block diagram of a fetal heart rate detection/tracking monitoring system in accordance with one embodiment of the invention.

FIG. 2 is a block diagram of a mechanical beam-steering system that can be used in the detection/tracking monitoring system shown in FIG. 1.

FIG. 3 is a detailed isometric illustration of one embodiment of the gimbal shown in FIG. 2.

FIG. 4 is a graphical representation of an example of the coverage of one embodiment of the beam-steering system shown in FIG. 2.

FIG. 5 is a block diagram of one embodiment of an electronic beam-steering system that can be used in the detection/tracking monitoring system shown in FIG. 1.

FIG. 6 is a graphical representation of ultrasonic pulses transmitted and received by one embodiment of the transducer shown in FIG. 1.

FIG. 7 is a graphical representation of an example of the coverage of one embodiment of the beam-steering system shown on FIG. 5.

FIG. 8 is a block diagram of an alternative transducer array that can be used in the electronic beam-steering system shown in FIG. 5.

FIG. 9 is a detailed block diagram of the detection/tracking monitoring system and mechanical beam-steering system shown in FIGS. 1 and 2.

FIG. 10 is a detailed functional block diagram of the control unit shown in FIG. 1.

FIG. 11 is a detailed block diagram of one embodiment of a signal processing algorithm that can implemented by the control unit shown in FIG. 1.

FIG. 12 is a detailed block diagram of one embodiment of a correlated noise removal algorithm that can be implemented as part of the signal processing algorithm shown in FIG. 11.

FIG. 13 is a detailed block diagram of one embodiment of a metrics calculating algorithm that can be implemented by the control unit shown in FIG. 1.

FIG. 14 is a block diagram of one embodiment of a pulse strength metric calculation algorithm that can be implemented as part of the metrics calculating algorithm shown in FIG. 13.

FIG. 15 is a state diagram of one embodiment of a pulse detection algorithm that can be implemented as part of the pulse strength metric calculating algorithm shown in FIG. 14.

FIG. 16 is a state diagram of one embodiment of a harmonic frequency metric detection algorithm that can be implemented in connection with a harmonic frequency metric calculating algorithm.

FIG. 17 is an illustration of a beam steering system in accordance with another embodiment of the invention.

### DETAILED DESCRIPTION OF THE PREFERRED EMBODIMENTS

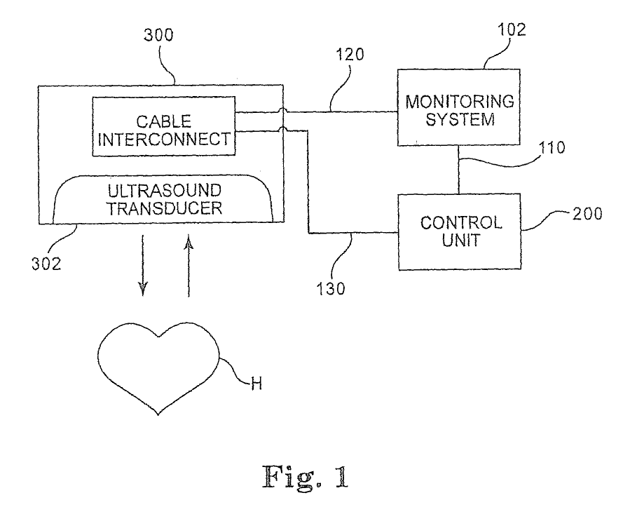

FIG. 1 is an illustration of a fetal heart rate detection/tracking monitoring system in accordance with one embodiment of the present invention. As shown, the system includes a commercially available fetal monitoring system 102 and ultrasound transducer 302 connected to a control unit 200 and beam-steering system 300. Monitoring system 102 and ultrasound transducer 302, which can, for example, be components of the Philips XM50 system, are connected by a cable 120. Although not shown in FIG. 1, straps or other mechanisms and approaches can be used to attach the beam-steering system 300 to a patient. These straps can, for example, be of a type similar to those that are known and commonly used to attach transducers such as 302 to patients.

Monitoring system 102 and transducer 302 operate in a conventional manner to produce and process ultrasonic pulses. These ultrasonic pulses are transmitted to a fetal heart H by the transducer 302 and are reflected by the heart back to the transducer. The reflected pulses received by the transducer 302 are coupled to monitoring system 102. Monitored pulse signals produced by the monitoring system 102 is coupled to the control unit 200 over cable 110. In one embodiment of the invention described below, the monitored pulse signals coupled to control unit 200 include the heart beat audio signal and the quality level signal produced by the monitoring system 102. Other embodiments (not shown) produce other signals representative of the pulses received by monitor 102. Control unit 200 includes stored data or metrics representative of detectable heart beat content in received transducer signals and associated search algorithms (not represented in FIG. 1). The pulse signals received from monitoring system 102 are processed and compared by control unit 200 to the stored metrics to assess the quality of the pulse signals. In response to this data processing operation, the control unit 200 executes tracking and search algorithms. Positioning commands are also generated and coupled to the beam-steering system 300 through cable 130. In response to the positioning commands the beam-steering system 300 will drive the ultrasound transducer 302 in such a manner as to optimize the quality of the ultrasonic signal received by the transducer (i.e., to maximize the heart beat-containing information in the pulse signals received by the transducer). This action is done dynamically, while the fetus moves within the womb. In effect, control unit 200 causes the beam-steering system 300 to dynamically find and/or track the moving fetal heart.

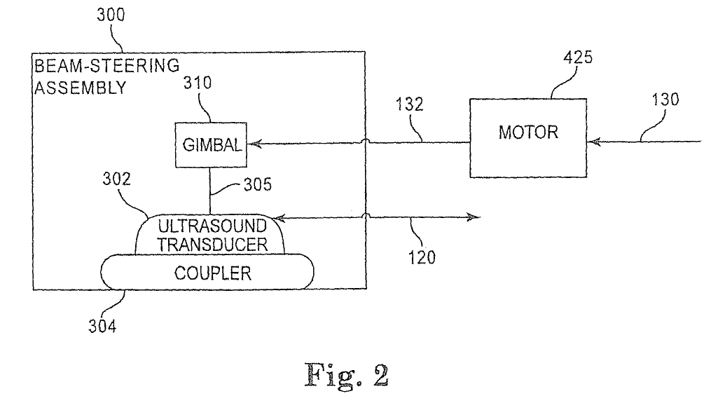

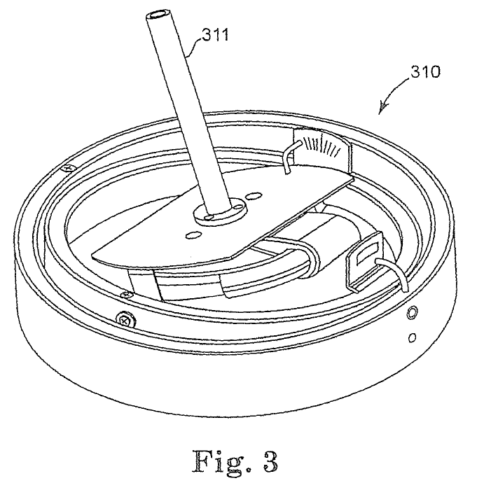

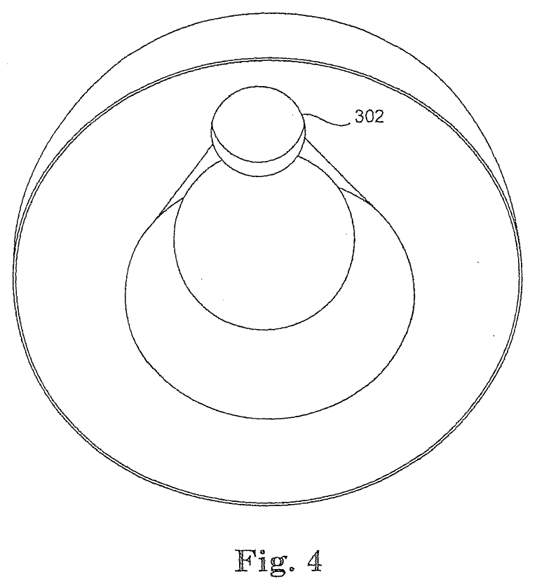

Beam-steering system 300 and control unit 200 can be implemented in a number of different ways. One embodiment of a mechanical beam-steering system 300 is shown in FIGS. 2-4. The mechanical beam-steering system 300 includes a mechanical apparatus such as a gimbal 310 to move the transducer 302 through tilting motions using a mechanical linkage 305. One embodiment of the gimbal 310 is shown in greater detail in FIG. 3. The illustrated embodiment of gimbal 310 includes a joystick 311 for enabling manual control over the positioning of the transducer 302. The transducer 302 is connected to the gimbal 310 by a mechanical linkage 305 (not shown in FIG. 3). In one embodiment the gimbal 310 can tilt the transducer 302 ±20° from horizontal in two axes. This tilting can, for example, provide a coverage cone such as that shown in FIG. 4, which will typically encompass at least 90% of expected fetal positions.

In the embodiment shown in FIG. 2 the transducer 302 is coupled to the patient's skin through a coupler 304. Coupler 304 provides a number of different functions. By way of example, the coupler 304 is transparent to the ultrasonic pulses produced and received by transducer 302. The coupler 304 is also compliant to allow the transducer 302 to tilt and maintain the maximum signal strength to and from the patient's skin.

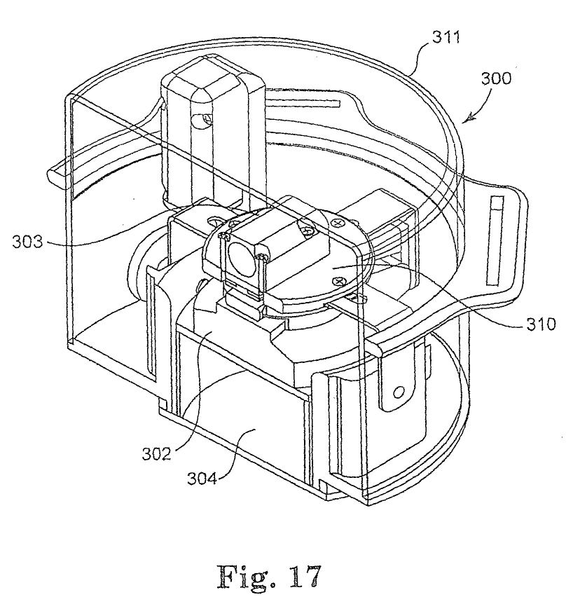

Another embodiment of the beam steering system 300 is shown in FIG. 17. Transducer 302, coupler 304 and gimbal 310 are mounted within a housing or enclosure 311 that is configured to be strapped or otherwise mounted to the patient. A pair of motors 303 are coupled to the gimbal 310 by gearing to provide tilting motion of the transducer 302. In one embodiment the motorized gimbal 310 can tilt the transducer 302 ±25° from horizontal in two axes. This tilting can, for example, provide a coverage cone such as that shown in FIG. 4, which can encompass at least 90% of expected fetal positions. Other embodiments of the invention are configured to tilt the transducer 302 within other angles (e.g., 5°-30°).

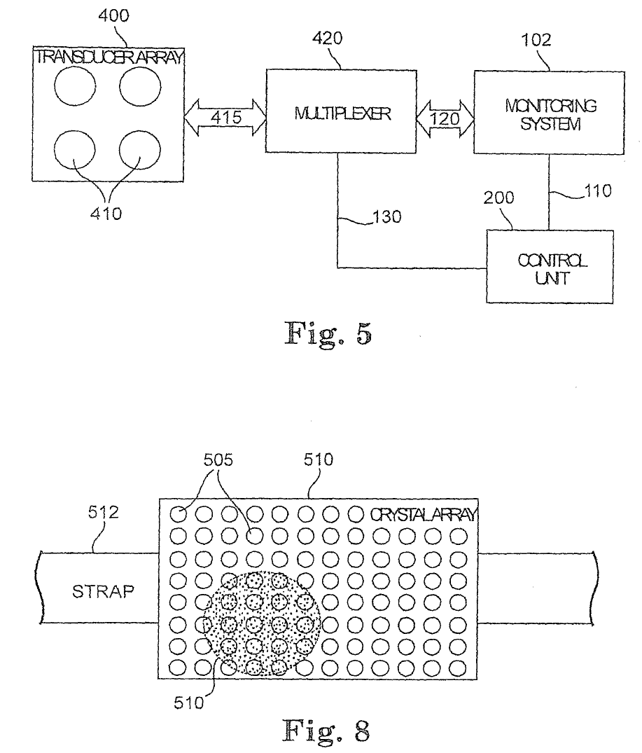

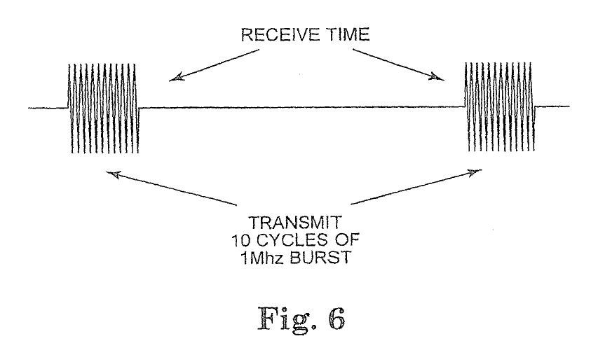

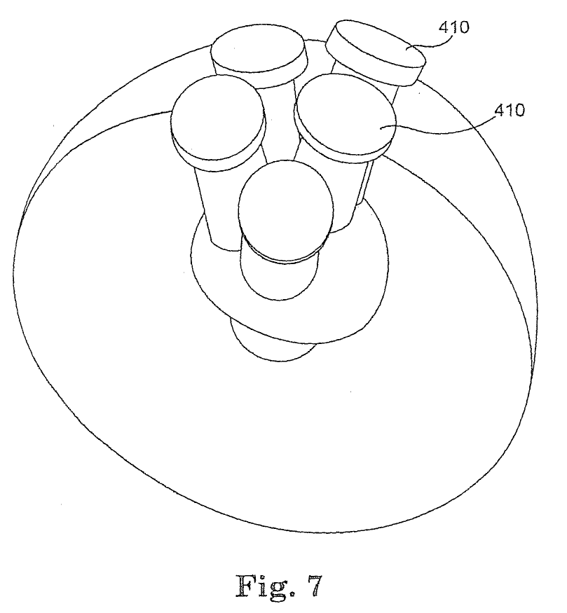

FIG. 5 is an illustration of one embodiment of an electronic beam-steering system 300. As shown, the electronic beam-steering system includes a transducer array 400 coupled to monitoring system 102 through a multiplexer 420 over cables 415 and 120, and to control unit 200 over cable 130. Transducer array 400 includes a plurality of individual ultrasound transducers 410, and can be a known and commercially-available device. Multiplexer 420 is commanded by control unit 200 to connect one or more of the transducers 410 to the monitoring system 102. Multiplexer 420, in combination with transducer array 400, effectively operates as an electronic beam-steering system in this embodiment of the invention. In one embodiment of the invention, only one transducer 410 would be enabled for transmitting at any one time. In this embodiment all the transducers 410 can be enabled during a receiving time period. A pulse timing sequence is illustrated generally in FIG. 6. Other groups of one or more transducers 410 are used for transmitting and/or receiving in other embodiments of the invention. FIG. 7 is a graphic illustration of an example of the coverage that can be provided by an electronic beam-steering system 300 of the type described above in connection with FIG. 5.

FIG. 8 is an illustration of a crystal array 500 can be used as an alternative to the transducer array 400 described above. Crystal array 500 includes a plurality of individual ultrasound crystals 505 which can be the same or similar to the crystals 410 described above. Crystals 505 are woven or otherwise mounted to a base such as a blanket that can be draped over the patient. The embodiment of crystal array 500 illustrated in FIG. 8 includes a strap 512 that can be used to secure the crystal array to the patient to minimize movement and maximize skin contact.

Any number of crystals 505 can be used to transmit ultrasonic pulses as long as the output power is within regulated limits. A selected subarray or cell 510 of crystals can be used for both ultrasonic signal transmitting and receiving. Multiplexer 420 can control the selection of crystals 505 to effectively “move” the cell 510 throughout the array 500. In another embodiment, all crystals 505 are used to receive the ultrasonic signal reflected from the patient for possible enhancement of the signal signal-to-noise ratio. In yet another embodiment of the invention a method of electrically steering the beam can be accomplished using a phased array approach. Still another embodiment can incorporate mechanically moving crystals 505, thereby steering the ultrasound beam. The mechanical movement can be accomplished by any number of approaches and structures—e.g. Magnetically, MEMs, piezo. This list is meant to be an illustration, not all-inclusive. These and other methods can be implemented within the transducer 302.

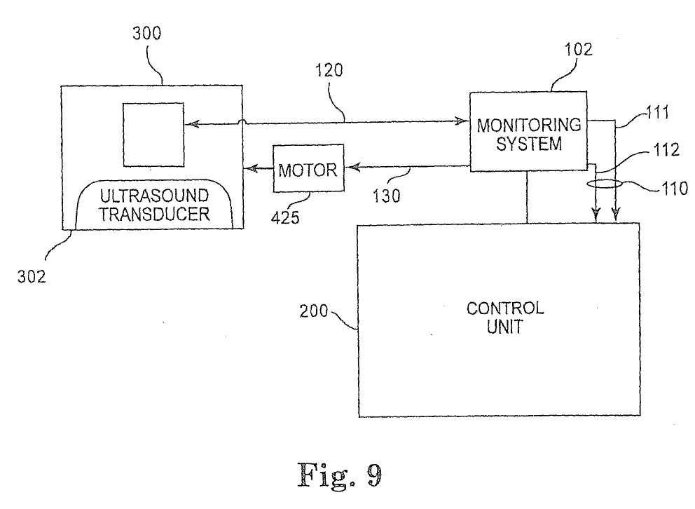

The interconnections between control unit 200 and monitoring system 102 are shown in greater detail in FIG. 9. As described above, in this embodiment the pulse signals processed by the control unit 200 to control the motion of beam-steering system 300 includes two signals provided by the monitoring system 102. One of these component signals is the audio signal of the fetal heart beat (represented at 111 in FIG. 9). The other component signal is the quality level signal (represented at 112 in FIG. 9). The quality level signal is a gross quality signal in this embodiment. It has three values and can be used to indicate an error condition if the received ultrasonic signal quality is deemed to be of insufficient quality. The heart beat audio signal and the quality level signal are processed by control unit 200 to ascertain the quality of the heat beat signal received by transducer 302, and to generate commands used to drive and position the beam-steering system 300 to optimize the quality of the received heart beat signal.

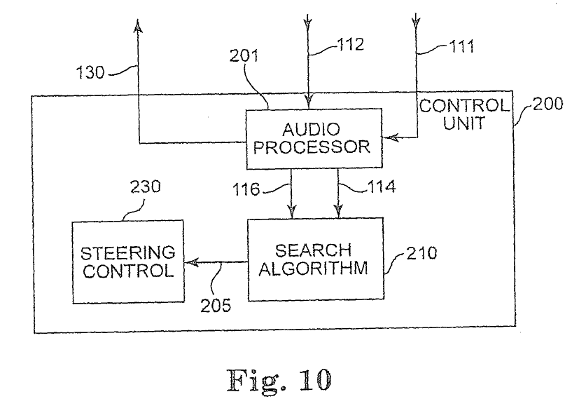

FIG. 10 is a functional block diagram of the control unit 200. As shown, the control unit 200 includes an audio processor 201, search algorithm 210 and steering control 230. The heart beat audio signal is processed by audio processor 201 to produce an audio quality signal. The audio processor 201 can use a digital signal processor (DSP) to break the signal into components. Non-limiting examples include wavelets, pulse shaping and spectrum plots, envelope analysis and combinations of these and/or other components. The audio quality signal is coupled to the search algorithm 210 over communication path 114. The quality level signal from the monitoring system 102 is coupled to the search algorithm 210 over communication path 116. In this embodiment of the invention, search algorithm 210 determines the quality of the fetal heart beat signal as a function of both the audio quality signal and the quality level signal. The fetal heart beat signal quality can, for example, be assessed by comparing the audio quality signal to the quality level signal. If the quality of the heart beat signal is too low, search algorithm 210 outputs commands to control the beam-steering system 300 (e.g., to motor 425 for the mechanical beam-steering assembly). In response, the position of the ultrasonic beam transmitted by the transducer 302 is changed. Search algorithm 210 can include programmed algorithms for determining possible search patterns. The objective of the search patterns is to keep the fetal heart beat signal quality from becoming too low (e.g., as determined by the quality signal received from the monitoring system 102). “Movement” of the transmitted and/or received portions of the ultrasonic beam can be achieved by the beam-steering system either physically (i.e., by mechanically moving the transducer 302) or electronically (i.e., by using different ultrasonic crystals of the arrays).

Any of a number of different methods can be performed by search algorithm 210. One method, known as Centering, periodically moves the transducer to identify the edge of the transducer beam at various angles. The width of the beam is then determined, and the transducer moved to the center of-the heart within this beam. Another method waits until the quality drops to a predetermined level. Then the Centering method could be used. Yet another method involves moving the beam until the quality reaches a certain minimum or “good enough” threshold level. Stored information representative of the previous locations of the fetal heart can also be used to generate the search routine. Neural networks or learning systems can also be used. A default routine can be used for startup or if and when any knowledge-based systems fail to locate the heart.

By way of example, two signal quality situations are presented. The first situation involves the complete loss of the fetal heart beat signal. In this situation the audio processor 201 will quickly identify the event on the basis of the audio quality signal. The event will typically be evident from the quality level signal shortly after it is evident from the audio quality signal. After identifying this event, search algorithm 210 will execute a search routine. On the basis of feedback provided by the received audio quality signal and the quality level signal, audio processor 201 and search algorithm 210 determine if the direction and amount of movement is correct (i.e. if the heart beat signal quality is increasing). Gross searching can cease, and tracking can be initiated, after the heart beat signal is identified to a sufficient quality level.

The other situation involves the relatively slow degradation and loss of the signal. In this situation search algorithm 210 can use the stored knowledge of past heart locations to determine the most effective search approach (e.g., the direction and amount of transducer beam movement). For example, the search routine could be executed in such a manner that it identifies the boundary of the transducer beam and center the heart within the beam. After the fetal heart is located, the search routine can cease.

Control unit 200 can also include a user override functionality (not shown in FIG. 10) that, when actuated by a clinician (e.g., by pressing a switch (not shown)) can cause the beam-steering system 300 to center the transducer within its range of motion. The override function can also stop any search routine that is being executed. Initial placement of the beam-steering system 300 on the patient can be facilitated by use of the override function. Use of the override function also allows the clinician to move the ultrasonic steering system to locate the fetal heart before operating the system in the automatic search or tracking modes. For example, the clinician could initiate the override functionality and place the beam-steering system 300 on the patient. After the optimum location is determined from the output of monitoring system 200, the beam-steering system 300 can be strapped or otherwise secured in place. The clinician could cause the control unit 200 to either operate in a tracking mode or to execute a search routine of the type described above to locate the fetal heart. The control unit 200 can also be configured to stop searching and issue an alarm (e.g., an audio alarm) if the fetal heart cannot be found within a predetermined period of time. Another use of an override function could be to allow the clinician to move the transducer 302 manually.

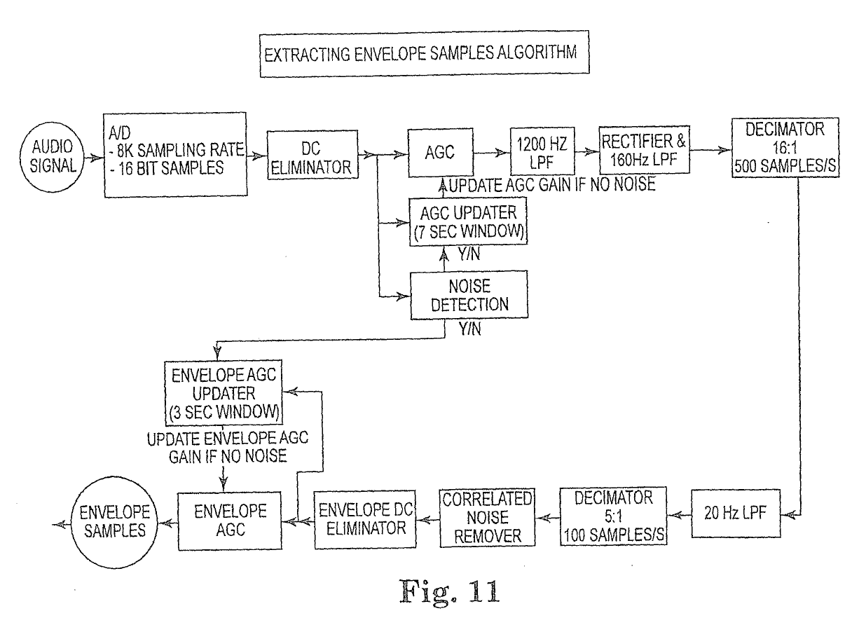

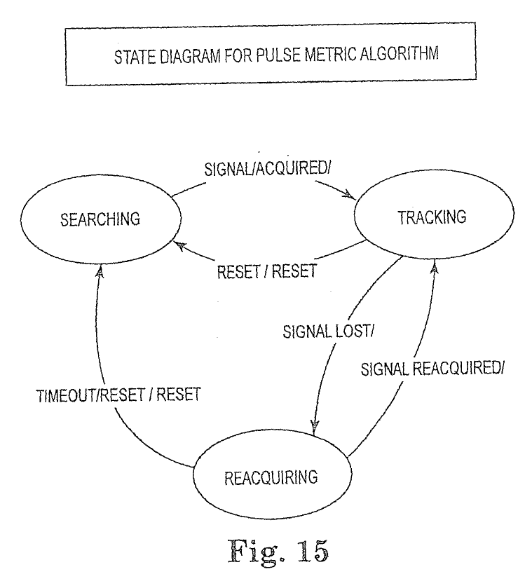

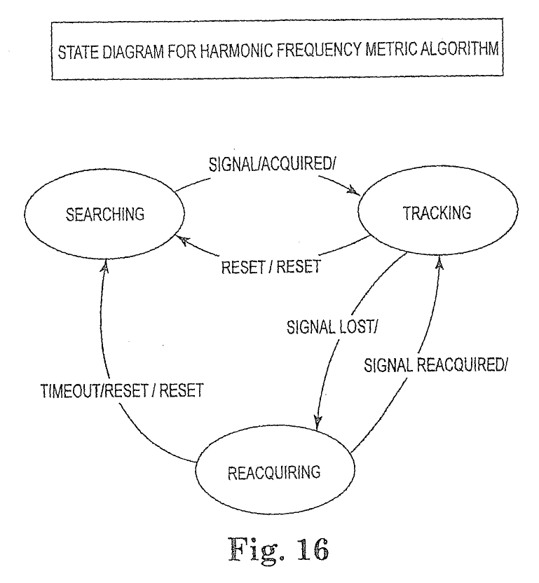

FIGS. 11-16 and the descriptions below are detailed block diagrams and descriptions of algorithms that can, for example, be implemented by control unit 200. In these embodiments the control unit 200 processes only the audio signal received from the monitoring system 200 (e.g., the signal shown at 111 in FIG. 9) to generate the control signals applied to beam-steering system 300). As shown in FIGS. 15 and 16, respectively, state processes are used in these algorithms in connection with the pulse strength metric calculation and the harmonic frequency metric calculation. The algorithms of FIGS. 11-16 and the following descriptions are provided only as an example. As is noted above, other algorithms can also be implemented by control unit 200.

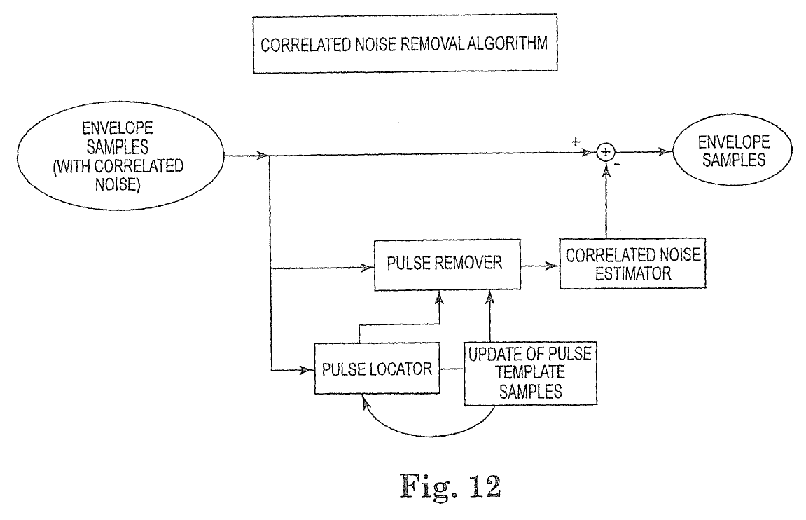

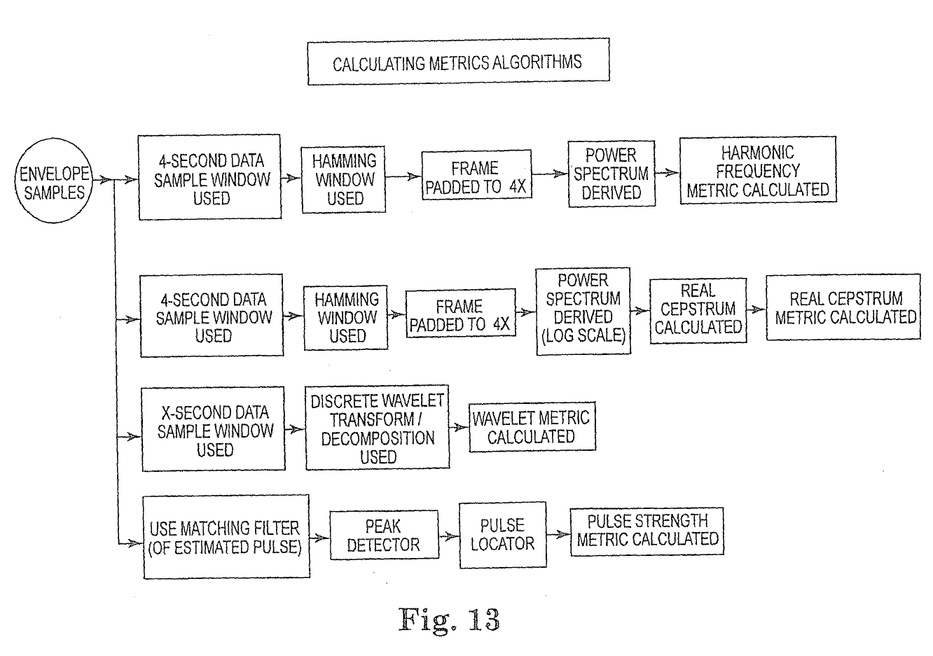

FIG. 11 illustrates an algorithm for extracting envelope samples. The audio signal can have a carrier greater than 500 Hz (seeking amplitude modulated pulse). The output of the AID can be 100 ms blocks. The output of the AGC can be constant power. The 1200 Hz LPF provides noise elimination. The rectifier and 160 Hz LPF operate as an envelope detector of the seeking amplitude modulated pulse. Additional details of the operation of the correlated noise remover are presented in the correlated noise removal algorithm shown in FIG. 12. The noise detection operation can use, for example, a 150 ms power spike window detector and a 300 ms power spike window detector. The envelope samples can be 100 samples/sec and 16 bit samples, and are provided to the calculating metrics algorithm shown in FIG. 13.

FIG. 12 illustrates a correlated noise removal algorithm. The envelope samples (with correlated noise) are those applied to the correlated noise remover of the envelope sample extraction algorithm shown in FIG. 11. The output of the pulse remover is essentially a residual signal, and the output of the correlated noise estimator is essentially correlated noise. Information representative of pulse locations is applied to the pulse remover from the pulse locator. The output of the update of pulse template samples to both the pulse remover and the pulse locator is a matching filter. Other parameters of the update of pulse template samples are 250 ms length and ARMA (AR-1) update of samples. The pulse locator uses a matching filter to locate pulses.

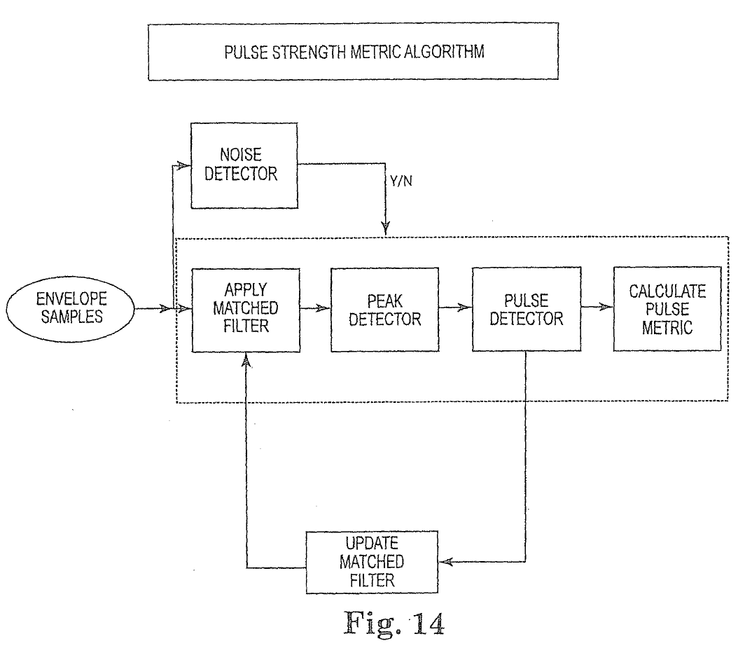

FIG. 13 illustrates an algorithm for calculating metrics. The envelope samples can be 100 16-bit samples/second. Metric options include the four flow paths for the envelope samples. The window of the 4-second data sample window used to calculate the harmonic frequency metric can move every 0.25 sec., and each data frame can be 400 samples. In the harmonic frequency metric calculation, the frame padded to 4× can include each data frame symmetrically padded to 1600 samples with 0s. The power spectrum derivation step can be done using 1/16 Hz resolution according to the formula: [Magnitude [FFT(1600 data points)]]². Other aspects of the harmonic frequency metric calculation are described in the state diagram for harmonic frequency metric shown in FIG. 16. The formula used to derive the power spectrum in the real cepstrum metric calculation can be: Log[Magnitude[FFT(1600 data points)]]. The formula used to calculate the real cepstrum can be: Magnitude[FFT(1600 power spectrum data points)]]. Other aspects of the pulse strength metric calculation are described in the pulse strength metric algorithm shown in FIG. 14.

FIG. 14 is an illustration of an algorithm for determining the pulse strength metric. The envelope samples can be 100 16-bit samples/second. Pulse detection and metric calculations can be paused when noise is detected by the noise detector. All local peaks can be detected by the peak detector. The pulse detector step can be based on current state—tracking or reacquiring/searching. Other aspects of the pulse detector step are described in the pulse strength metric state diagram shown in FIG. 15. Other parameters of the update matched filter are 250 ms length and ARMA (AR-1) update of matched filter coefficients.

FIG. 15 is an illustration of a state diagram for the pulse metric algorithm. In connection with the searching state, searches are done for 3 consecutive pulses in the 1-4 Hz range, and the pulses must be equally spaced. Local maximums from the output of the matching filter are found. Metric values for equally spaced local maximums found between 1 and 4 Hz are calculated. If the 3-pulse metric is greater than the tracking threshold, the operation returns to the tracking state. Inter-pulse distance (1/pulse rate) is determined and used in the tracking state. In connection with the tracking state, searches are done for the next pulse base on the estimated inter-pulse distance and the two previous pulse locations. Local maximums from the output of the matching filter are used to locate the largest maximum in the time window where the next pulse should be located. The window size can be ±10% of the inter-pulse interval (1/pulse rate). The largest maximum (that will give the largest metric) is chosen. Current inter-pulse distance (pulse rate) is updated. If the metric value falls below the minimum tracking threshold, the operation returns to the reacquiring state. In connection with the reacquiring state, searches are done for three consecutive pulses. Three-pulse metric values for pulse combinations at the last known current pulse rate ±20% are calculated. The pulses must be equally spaced. Local maximums from the output of the matching filter are found. The pulse combination with the largest metric is chosen. If the metric is greater than the tracking threshold, the operation returns to the tracking state. Inter-pulse distance (pulse rate) is determined and used in the tracking state.

An example of a harmonic frequency metric algorithm follows. The harmonic frequency metric is a measurements of the strength of the frequency harmonics caused by a periodic pulse (pulse train). It is a measurement of: Power of the first four harmonics/Total signal power. Signal power is constant, so it can be ignored. The frequency resolution is 1/16 Hz, so the fundamental and harmonic locations are at multiples of 1/16 Hz, or N 1/16 Hz where N is the harmonic number.

In the following equations, the locations of harmonics will be specified at the integer (also called the bucket) location in the frequency domain. The discrete location of the peak of the fundamental frequency or one of its harmonics is called “Location F” in the following algorithm. Then, the power of each harmonic (or the fundamental frequency) is calculated as: Harmonic Power=Amplitude (Location F)+Amplitude (Location F−1)+Amplitude (Location F+1). The base metric is calculated as follows: Metric=Fundamental Freq. Power+1^{st} Harmonic Power+2^{nd} Harmonic Power+3^{rd} Harmonic Power.

An example of an algorithm to compensate for fundamental frequency and harmonics not centered on a discrete frequency follows. The fundamental frequency is seldom actually centered exactly at N 1/16 Hz. That is one of the reasons for summing the adjacent locations to the peak harmonic location when calculating the power of the harmonic. This lack of centering also results in the harmonics not being located at exactly a multiple of the fundamental frequency location (as would be true for an analog signal). By considering the possible true analog offsets of the fundamental frequency, it has been derived that there are nine possible combination locations for the harmonics that need to be considered for one fundamental frequency location. The metric algorithm considers all nine possible combinations (called base metrics) and chooses the largest value. If the location of the fundamental is at location F, Table 1 below shows the possible combinations that are used.

TABLE 1

| | Fundamental | | | |
|---|---|---|---|---|
| Combi- | Frequency | 1^{st} Harmonic | 2^{nd} Harmonic | 3^{rd} Harmonic |
| nation # | Location | Location | Location | Location |
| 1 | F | 2F − 1 | 3F − 2 | 4F − 2 |
| 2 | F | 2F − 1 | 3F − 1 | 4F − 2 |
| 3 | F | 2F | 3F − 1 | 4F − 1 |
| 4 | F | 2F | 3F | 4F − 1 |
| 5 | F | 2F | 3F | 4F |
| 6 | F | 2F | 3F | 4F + 1 |
| 7 | F | 2F | 3F + 1 | 4F + 1 |
| 8 | F | 2F + 1 | 3F + 1 | 4F + 2 |
| 9 | F | 2F + 1 | 3F + 2 | 4F + 2 |

FIG. 16 is an illustration of a state diagram for the harmonic frequency metric algorithm. In connection with the searching state, searches are done for the fundamental frequency peaks. The metric value for each local maximum found between 1 and 4 Hz is calculated. The peak for the fundamental frequency that generates the highest metric is chosen. Operation returns to the tracking state if the metric is greater that the tracking threshold. In connection with the tracking state, the metric value for the current fundamental frequency location is calculated. The metric value for adjacent frequency locations is also calculated, and operation will switch to an adjacent frequency location if the metric value of the neighbor is higher. If the metric value falls below the minimum tracking threshold, operation returns to the reacquiring state. An description of the metric calculation for the tracking state is provided above in the description of the harmonic frequency metric algorithm. In connection with the reacquiring state, the metric values for the last known current fundamental frequency location and for adjacent frequency locations are calculated. The location with the largest metric value is chosen. If the metric is greater than the tracking threshold, the operation returns to the tracking state. If the metric has not exceeded the tracking threshold after a specific period of time (e.g. 10 sec), the operation returns to the searching state.

Although the above description refers to the audio signal available from the monitor, other embodiments of the invention to implement the above algorithms within the monitor itself, before other processing of the signal has occurred.

Although the present invention has been described with reference to preferred embodiments, those skilled in the art will recognize that changes can be made in form and detail without departing from the spirit and scope of the invention. In particular, although described in connection with an ultrasonic monitoring system, the invention can be used in connection with other heart beat monitoring modalities, including those that do not require the transmission of a signal in connection with the monitoring of the heart beat. For example, the invention can be used to control the orientation of a sensitive, unidirectional (e.g., parabolic) microphone that receives an audio signal produced by a heart. In other embodiments, only one of either a transmitter and a receiver is controlled by the steering control system.

What is claimed is:

1. A heart rate monitoring system, including:
a transducer for transmitting and/or receiving heart beat monitoring signals;
a steering system for controlling the location of the transmitted and/or received heart beat monitoring signals;
a monitoring system coupled to the transducer for processing the received heart beat monitoring signals and for producing heart beat information representative of a monitored heart beat;
a control unit coupled to the monitoring system and the steering system, for processing the received heart beat monitoring signals and controlling the steering system to optimize quality of the heart beat monitoring signals.

2. The heart rate monitoring system of claim 1 wherein the control unit processes the received heart beat monitoring signals to optimize the quality of the heart beat information in the heart beat monitoring signals.

3. The heart rate monitoring system of claim 1 wherein the steering system includes a mechanical steering system.

4. The heart rate monitoring system of claim 3 wherein the steering system includes a mechanical steering system connected to the transducer.

5. The heart rate monitoring system of claim 1 wherein the steering system includes an electronic steering system.

6. The heart rate monitoring system of claim 5 wherein:
the transducer includes an array of transducer elements; and
the electronic steering system selects the transducer elements.

7. The heart rate monitoring system of claim 6 wherein the electronic steering system includes a multiplexer coupled between the control unit, the monitoring system and the array of transducer elements.

8. The heart rate monitoring system of claim 1 wherein the control unit is operable in a search mode and a tracking mode.

9. The heart rate monitoring system of claim 1 wherein the control unit includes a search algorithm and a steering controller.

10. The heart rate monitoring system of claim 1 wherein the control unit is operable in a manual override mode.

11. The heart rate monitoring system of claim 1 wherein:
the monitoring system produces a plurality of signals representative the monitored heart beat; and
the control unit controls the steering system as a function of the plurality of signals.

12. The heart rate monitoring system of claim 11 wherein the plurality of signals produced by the monitoring system include an audio signal and a quality level signal.

13. A fetal heart rate monitoring system, including:
an ultrasonic transducer for transmitting a signal to a patient and for receiving a reflected heart beat monitoring signal;
a monitoring system coupled to the ultrasonic transducer, for processing the heart beat monitoring signal and producing heart beat information representative of the monitored heart rate;
a steering system coupled to the ultrasonic transducer, for controlling the location of signal transmitted to the patent and/or the location of the received heart beat monitoring signal with respect to the patient; and
a control system coupled to the monitor and steering system, for controlling the steering system as a function of the heart beat monitoring signal to optimize the quality of the received heart beat monitoring signals.

14. The fetal heart rate monitoring system of claim 13 wherein:
the steering system is a mechanical steering system; and
the control system causes the mechanical steering system to move to optimize the quality of the received heart rate monitoring signals.

15. The fetal heart rate monitoring system of claim 13 wherein:
the ultrasonic transducer includes an array of ultrasonic elements;
the steering system is an electronic steering system; and
the control system causes the electronic steering system to select elements in the array of ultrasonic elements.

16. The fetal heart rate monitoring system of claim 13 wherein:
the monitoring system processes the heart rate monitoring signal to produce a plurality of signals including information representative of the quality of the heart beat-related information contained in the heart rate monitoring signals; and
the control unit controls the steering system as a function of the plurality of signals produced by the monitoring system.

17. The fetal heart rate monitoring system of claim 16 wherein the plurality of signals produced by the monitoring system include an audio heart rate signal and a quality level signal.

18. The fetal heart rate monitoring system of claim 13 wherein the control unit is operable in search and tracking modes.

19. The fetal heart rate monitoring system of claim 13 wherein the system is operable in an override mode.

20. Components for use in connection with an ultrasonic fetal heart rate monitoring system of the type having an ultrasonic transducer and a monitor, the transducer for transmitting a signal to a patient and for receiving a reflected heart rate monitoring signal and the monitor for processing the heart rate monitoring signal and producing information representative of the monitored hear rate, the invention including:
a steering system for connection to the ultrasonic transducer, for controlling the location of a signal transmitted to the patent and/or the location of a received heart rate monitoring signal with respect to the patient; and
a control system for connection to the monitor and steering system, for controlling the steering system as a function of the heart rate monitoring signal to optimize the quality of the received heart rate monitoring signals.

21. The method of claim 20 wherein controlling the steering system includes optimizing the quantity of heart beat-related information in the heart rate monitoring signal.

22. A method for monitoring a fetal heart rate, including:
operating a transducer to receive a heart rate monitoring signal;
monitoring the quality of the heart rate monitoring signal; and
controlling the transducer as a function of the heart rate monitoring signal to optimize the quality of the heart rate monitoring signal.

23. The method of claim 22 wherein controlling the transducer includes controlling the location of signals transmitted by the transducer and/or the location of the heart rate monitoring signals received by the transducer.

24. The method of claim 23 wherein controlling the location of signals includes mechanically controlling the position of the transducer.

25. The method of claim 23 wherein:
operating the transducer includes operating an array of transducer elements; and
controlling the location of signals includes electronically selecting transducer elements of the array.

26. The method of claim 22 wherein controlling the transducer includes optimizing the quantity of heart beat-related information in the heart rate monitoring signal.
